(19) United States
(12) Patent Application Publication
(10) Pub. No.: US 2023/0122683 A1
(43) Pub. Date: Apr. 20, 2023

(54) HYDROGELS FOR CULTURED MEAT PRODUCTION

(71) Applicant: Mosa Meat B.V., Maastricht (NL)

(72) Inventors: Arin DOGAN, Maastricht (NL); Mark POST, Maastricht (NL); Sergio SPAANS, Maastricht (NL)

(21) Appl. No.: 17/759,314

(22) PCT Filed: Feb. 3, 2021
(86) PCT No.: PCT/NL2021/050068

§ 371 Date: Jul. 22, 2022

(30) Foreign Application Priority Data
Feb. 3, 2020 (NL) 2024820

(51) Int. Cl. C08B 37/00; C12N 5/077; C12N 5/00

(52) U.S. Cl. CPC C08B 37/0084; C12N 5/0659; C12N 5/0062; C12N 2533/74; C12N 2513/00; C12N 2533/50

(57) ABSTRACT

The invention is directed to a modified polysaccharide hydrogel, comprising a low molecular weight alginate with a specific M/G ratio. The modified polysaccharide is modified with a specific peptide, preferably comprising a cell-adhesion peptide. The modified polysaccharide hydrogel may be used as a hydrogel for the growth of cultured meat preferably as a sacrificial biopolymer.

The invention is in the field tissue generation, in particular in the field of cultured meat. In particular the invention is related to a hydrogel, in particular a hydrogel environment for growing tissue, a method to produce said hydrogel and use of said hydrogel for the production of cultured meat.

A hydrogel is a network wherein the continuous phase is solid and the discontinuous phase is water. The continuous phase is a network of hydrophilic polymer chains. The polymer chains are crosslinked, resulting in a certain structural integrity. Crosslinks may be physical or chemical. Physical crosslinks include hydrogen bonds, hydrophobic interactions, chain entanglement. Chemical crosslinks are based upon covalent bonds between polymer strands. The structural integrity of the polymer network remains intact, so it does not dissolve or collapse, by addition of water. Moreover, a hydrogel is capable of absorbing water to a high extent. Water can be present in over 90 wt %, and for instance in alginate-based hydrogels for more than 99.5 wt %.

Hydrogels are versatile as they may be biodegradable, biocompatible and non-toxic. For example, uses are found as drug delivery systems or as media in tissue engineering. In tissue engineering the hydrogels mimic the natural 3D environment of cells.

Tissue generation is a process of renewal and growth to repair or replace tissue. A related term to tissue generation is regenerative medicine, which deals with replacing, engineering or regenerating cells, tissues and organs to restore or establish normal function. It also includes the possibility to create tissue and organs ex vivo from parent cells. The type of parent cell is chosen depending on the function of the final tissue or organ. Organ transplant rejection may be limited if regenerative medicine is used wherein the parent cell is derived from the patient. Besides using tissue generation for a medical purpose, tissue generation can find purpose in e.g. cultured meat, as an alternative to the traditional meat for consumption.

Meat for human consumption is generally muscle tissue from animals such as cows, pigs, sheep and the like. The cells making up the muscle tissue originate from parent cells, in particular precursor cells called myosatellite cells and multipotent adult stem cells. Myosatellite cells are multipotent and can proliferate and differentiate to become a plurality of specialized cells.

A discussion remains on the environmental impact and animal cruelty in the meat industry. The environmental impact of the meat industry is associated with the animal methane production, effluent waste, water and land consumption. The animal welfare issues are related to the handling of live animals, such as the amount of received daylight, or the surface of land per animal. An increasing number of people have become vegetarian or vegan, partly due to the negative impact of the meat industry. Therefore, a demand has arisen for alternatives to meat.

Cultured meat is one of the alternatives that has been proposed. Cultured meat is produced from myosatellite cells that are induced to grow into muscle tissue. The growth process includes migration, spreading, guidance, proliferation, differentiation of the cells and takes place ex vivo. The myosatellite cells may be obtained without the need to slaughter animals. Engineered muscle tissue constructs may be harvested and used for human consumption.

In nature the extracellular matrix (ECM) is responsible for various aspects in the lifecycle of the cells, including proliferation and differentiation. ECM is viscoelastic, with properties of both viscous liquids and elastic solids. The composition of ECM is broadly classified as the combination of water, minerals, proteoglycans and fibrous proteins. The final function of the obtained cells or tissue is determined by the chemical, topographical and mechanical properties of the ECM. It is therefore typically required that synthetic biomimetic environments provide similar properties. Synthetic ECM structures may comprise proteins or polysaccharides, such as alginate.

Alginate is an anionic biopolymer comprising α-L-guluronate (also referred to as G), and β-D-mannuronate (also referred to as M). It is a versatile material and it finds its purpose in areas such as food additives, pharmaceutics, dentistry and bioengineering. Moreover, alginate is capable of crosslinking in the presence of divalent cations, such as Ca²⁺, Mg²⁺, resulting in a network that may be used to encapsulate materials.

Alginate is often used in bioengineering as it is biocompatible and non-toxic. It encourages cell proliferation and mammalian cells do not express enzymes that can degrade the polysaccharide. However, a drawback is the lack of structural stability and lack of cellular interactions to effectively guide cellular alignment needed to produce tissue. Often collagen is added to provide structural stability and collagen can provide the necessary cellular interaction. However, collagen is animal derived and obtained through methods that cause harm in animals and thus contradicts to the purpose of cultured meat.

Chaudhuri et al. (Nature Materials. 15, (2016), 326-336) describe the use of modified-alginate hydrogels for 3D cell cultures to promote tissue regeneration. The modification of the alginate includes covalent coupling to integrin-binding ligands to promote cell adhesion. Such an integrin-binding ligand is the peptide motif RGD (Arginine-Glycine-Aspartic acid). RGD is naturally found in the extracellular matrix and it is typically considered the most common motif responsible for cell adhesion as the RGD sequence is recognized by integrins. Integrins are transmembrane receptors of a cell. Chaudhuri et al. describe that tuning the rate of stress relaxation of the modified-alginate hydrogels impacts the cell spreading, proliferation and osteogenic differentiation of mesenchymal stem cells. However, the hydrogel for osteogenic differentiation is not directly usable for myogenic differentiation, as the chemical, topographical and mechanical properties of the growth milieu are not compatible with the specific needs of the satellite cells.

WO2018136012 describes a modified alginate copolymer with grafted moieties to the alginate backbone. The grafted moieties comprise a polymer and a stabilizing group. A drawback of such modified-alginate is the lack of suitable conditions for cells to align and form compacted engineered muscle constructs. Compacted engineered muscle constructs can be described as an increase in cell-matrix density, which is typically considered crucial for the formation of muscle tissue.

WO 98/12228 describes materials containing polymers comprising polysaccharides such as alginates or modified alginates that may be used for tissue engineering applications. The alginates may be modified by covalent bonding to a biologically active molecule for cell adhesion or other cellular interactions. However, the materials described therein do not provide optimal structural and mechanical properties for an environment for the production of muscle tissue.

Baker et al. (PNAS, 109, (2012), 14176-14181) describe biomaterial scaffolds for templates for directed formation of functional tissue. The biomaterial scaffolds comprise poly(ε-caprolactone) and poly(ethyleneoxide), wherein poly(ethyleneoxide) serves as sacrificial element. The poly(ethyleneoxide) directly dissolves upon hydration, thus being a moiety that can be selectively removed. However, a lack of structural integrity results from the instantaneous elimination of the sacrificial element.

It is an object of the present invention to provide a hydrogel which in part overcomes the above mentioned drawbacks.

The present inventors have found that polysaccharides, in particular alginate, can be selected and/or modified so that they become suitable as hydrogel (functioning as a scaffold) to encapsulate cells for the production of cultured meat. Surprisingly, this is achieved by a modified polysaccharide, in particular alginate, with a specific molecular weight (M_{w}) and a specific composition, resulting in the provision of suitable circumstances for the formation and/or growth of muscle cell tissue.

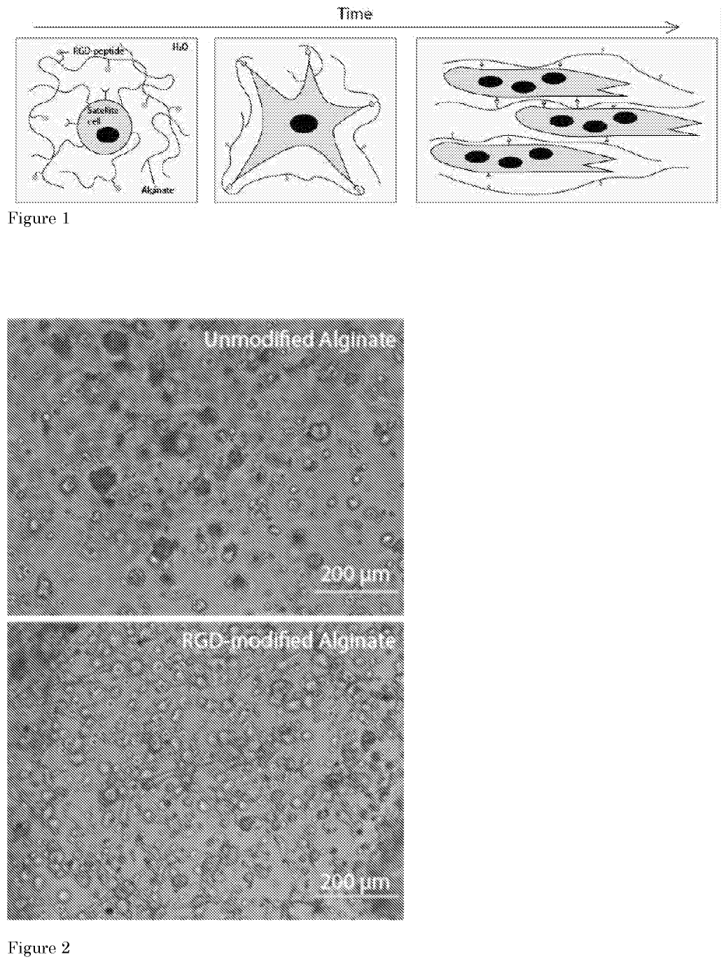

FIG. 1 is a schematic overview of a RGD modified-alginate allowing cells to find each other, spread and form aligned morphologies.

FIG. 2 shows microscope images that depict the change in cellular shape and dispersion in 3D between unmodified alginate and RGD-modified alginate after 2 days.

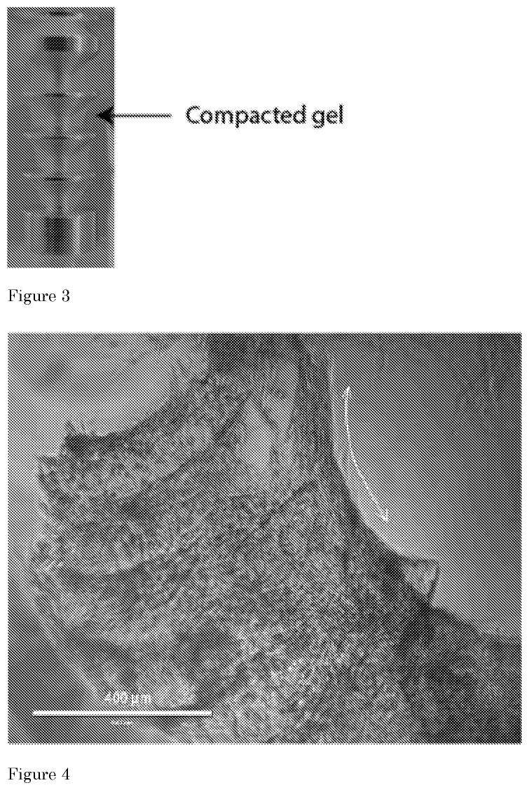

FIG. 3 illustrates a pillar used to form compacted hydrogels, which result in the formation of muscle tissue.

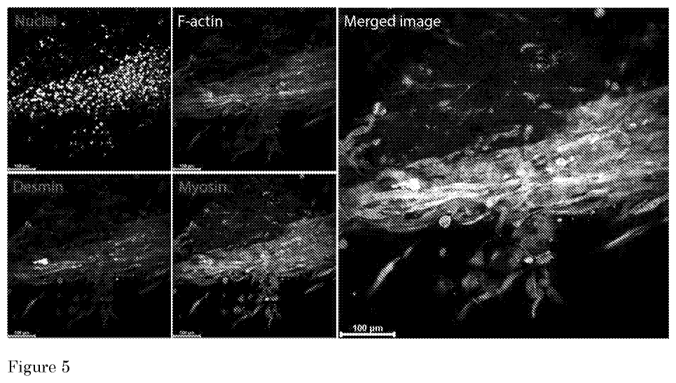

FIG. 4 is a microscope image showing the alignment of myosatellite cells in the compacted hydrogel.

FIG. 5 shows immunofluorescent images indicating the alignment of myosatellite cells, formation of multinucleated cells and the expression of myosin, filamentous-actin, desmin and nuclei in RGD-modified alginates.

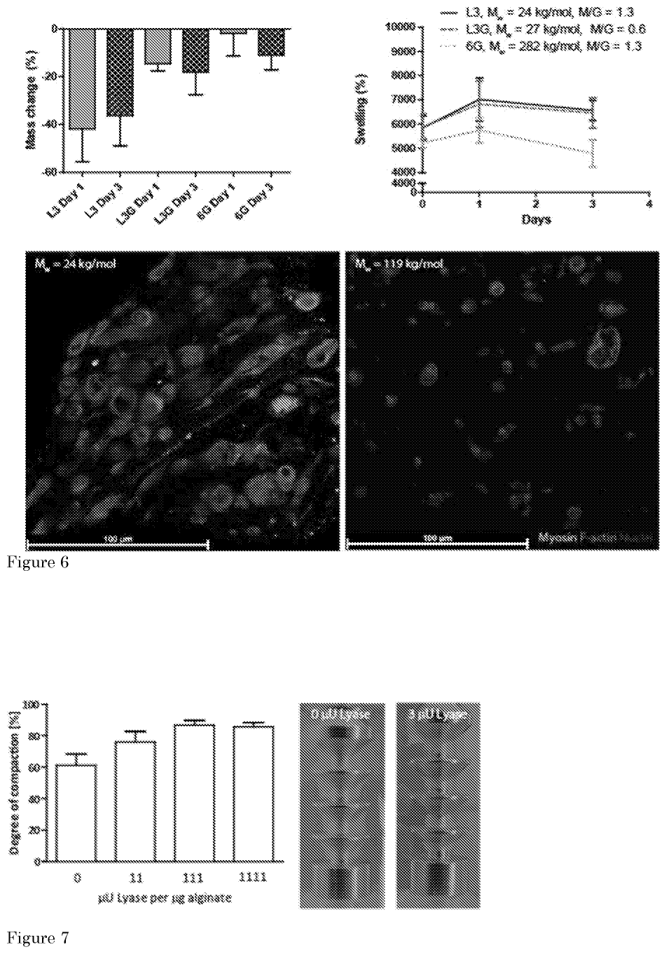

FIG. 6 shows the increased biodegradability and swelling of low molecular weight alginate and resulting effect on cellular alignment.

FIG. 7 shows the effect of enzymatic degradation, using alginate lyase, on the degree of compaction of the hydrogels.

Thus, in the first aspect, the present invention is directed to a modified polysaccharide hydrogel comprising a low molecular weight alginate with a molecular weight of 10 to 50 kDa and a M/G ratio of 0.8 to 1.5, wherein the alginate is conjugated with one or more cell-adhesion peptides.

The low molecular weight of the alginate typically allows for increased biodegradability which may be beneficial for removal through metabolic functions e.g. the kidneys. More importantly, the low molecular weight of the modified alginate is typically preferred as it allows for a faster stress relaxation, due to the shorter chain length. A faster stress relaxation generally allows myosatellite cells to spread within the gel, as can be seen in FIG. 6. The faster stress relaxation further typically allows for the myosatellite cells to form cell-cell contacts and myotubes. Accordingly, the low molecular weight alginate may provide a hydrogel with structural and mechanical properties that is typically an optimal environment for the production of muscle tissue, and therefore cultured meat.

In accordance with the invention, the modified polysaccharide hydrogel, comprising alginate, is used to encapsulate cells. Alginate is isolated from seaweed and different seaweed species, each resulting in a specific molecular weight and composition. In accordance with the invention the alginate is typically from food grade sources, to allow for use for cultured meat for consumption. With food grade sources is meant any material safe for human consumption complying to the Food Chemicals Codex or any equivalent standard. Further, the alginate is conjugated with one or more cell-adhesion peptides, such as RGD. In the hydrogel of the present invention the cells may differentiate and mature.

In a preferred embodiment the alginate is modified with a first specific peptide, which is preferably animal-free. Preferably, the specific peptide comprises a cell-adhesion peptide. The cell-adhesion peptide is capable of binding to a receptor on the cell to encourage several processes, such as cell migration, spreading, guidance, proliferation and differentiation. Cell adhesion peptides may attach to various integrin receptors on the cell surface. They induce attachment, signaling and remodeling through cleavage.

It is also possible to provide a plurality of different cell-adhesion peptides in the hydrogel. Accordingly, the alginate may be modified by two or more different cell-adhesion peptides such as RGD and another functional peptide such as GGGGDGEA, which is considered relevant for satellite cell interaction. In this peptide sequence, the independent letters correspond to a specific amino acid (e.g. G is glycine). The use of two or more cell-adhesion peptides typically allows for a plurality of binding sites, which may result in increased encouragement of migration, spreading, guidance, proliferation and differentiation. Moreover, an additional carrier or support affects the chemical, topographical and mechanical properties of the modified alginate, which is related to the final function of the tissue.

More preferably, the specific peptide comprises an integrin-binding ligand. Integrins, upon binding to integrin-binding ligands, activate signal transduction pathways that mediate cellular signals, including regulation of the cell cycle. Regulation of the cell cycle includes processes such as cell spreading, migration, guidance, proliferation, apoptosis. Integrins are moreover responsible for tissue organization, hemostasis, inflammation, target recognition of lymphocytes, differentiation of cells by the interaction of the integrin with the environment.

Examples of suitable integrin-binding ligands are for instance given by Humphries et al. (J. Cell Sci. 119 (2006) 3901-3903) and comprise fibronectin, osteopontin, laminin, collagen, ADAM family members, COMP, connective tissue growth factor, Cyr61, E-cadherin, fibrillin, fibrinogen, ICAM-4, LAP-TGFβ, MMP-2, nephronectin, L1, plasminogen, POEM, tenascin, thrombospondin, VEGF-C, VEGF-D, vitronectin, heparin and combinations thereof.

Preferably the specific peptide comprises cell-adhesion peptides, more preferably RGD. RGD is naturally found in the extracellular matrix and it is considered the most common motif responsible for cell adhesion. FIG. 1 is a schematic overview of a preferred embodiment wherein a RGD modified-alginate allows cells to find, spread and form aligned morphologies. FIG. 2 shows micrographs where the difference of cell shape after two days between the cells in an unmodified alginate hydrogel and a RGD modified alginate hydrogel according to a preferred embodiment of the present invention is visualized.

In a preferred embodiment of the invention, the alginate is crosslinked. Crosslinking is achieved via cations, as the alginate is an anionic polymer. The concentration in which the cations are present determines the crosslinking density. Preferably, the concentration of the cations for crosslinking during preparation of the hydrogels is between 0.05 to 0.5 M. When the hydrogels are used with a cell culture it may also be desirable to have these cations present, in which case the concentration of cations is preferably between 0 to 50 mM. The crosslinking density is in part responsible for the rigidity of the system, thereby having an influence on the chemical, topographical and mechanical properties of the modified-polysaccharide hydrogel. The chemical, topographical and mechanical properties determine the final function of the tissue. The preferred concentration of the cations and thus the degree of crosslinking typically presents suitable chemical, topographical and mechanical properties for myosatellite cells to migrate, spread, align, proliferate and differentiate into muscle tissue. Preferably, the cations are divalent cations. More preferably, the divalent cations are calcium ions (Ca²⁺).

In a preferred embodiment, the alginate present in the modified polysaccharide hydrogel comprises one or more further specific peptides, wherein said one or more further specific peptides are different from the other specific peptide. The further specific peptide may function as an additional carrier and/or support for the cells.

Preferably, the further specific peptide also comprises a cell-adhesion peptide.

More preferably the further specific peptide comprises an integrin-binding ligand. Integrins are transmembrane receptors that, upon binding to integrin-binding ligands, activate signal transduction pathways that mediate cellular signals, including regulation of the cell cycle. Processes such as cell spreading, migration, guidance, proliferation, and apoptosis are all directly or indirectly related to the regulation of the cell cycle. Integrins are moreover responsible for tissue organization, hemostasis, inflammation, target recognition of lymphocytes, and differentiation of cells by the interaction of the integrin with the environment.

As mentioned above, suitable examples of integrin-binding ligands are fibronectin, osteopontin, laminin, collagen, ADAM family members, COMP, connective tissue growth factor, Cyr61, E-cadherin, fibrillin, fibrinogen, ICAM-4, LAP-TGFβ, MMP-2, nephronectin, L1, plasminogen, POEM, tenascin, thrombospondin, VEGF-C, VEGF-D, vitronectin, heparin (Humphries et al. (J. Cell Sci. 119, (2006), 3901-3903)).

Most preferably the specific peptide comprises RGD.

It may be appreciated that the modified polysaccharide hydrogel may be used for the promotion of muscle tissue regeneration, preferably as a sacrificial biopolymer. The term sacrificial is used herein to describe the possibility to selectively remove the biopolymer from the tissue. Selective removal may be achieved by dissolving the polysaccharide either via diffusion, using a chelator (e.g. EDTA) and/or enzymatic degradation (e.g. Alginate Lyase) of the polysaccharide. FIG. 7 shows the effect of alginate lyase on the degree of compaction and accordingly illustrates the increase of cell-matrix density via selective degradation of alginate. As a result of the selective degradation, the degree of compaction may be controlled. The modified polysaccharide hydrogel—typically provides an environment for several processes such as cell guidance, spreading, migration, proliferation and differentiation. The processes are necessary during the regeneration process of cells and thus for the regeneration of tissue. A damaged tissue may be encouraged to regenerate by the modified polysaccharide hydrogel. The modified polysaccharide hydrogel according to the present invention thus typically provides a suitable environment for the regeneration process of tissue.

The modified polysaccharide hydrogel according to the present invention, more importantly, further provides an ideal environment for the production of cultured meat. The modified polysaccharide hydrogel may accordingly be used in the production of cultured meat suitable for consumption, preferably as sacrificial biopolymer.

As the cultured meat is suitable for consumption it is typically required that there are substantially no toxic compounds present in the modified polysaccharide hydrogel. Toxic is herein defined as considered non-safe for human consumption. Therefore, it is typically preferred that the purity of the modified polysaccharide is at least complying to such as, but not limited to, the purity criteria in a European Council Directive, such as the Directive concerning food additives other than colours and sweeteners authorized for use in foodstuffs for human consumption. Further, it may be beneficial to add nutrients to the hydrogel such as vitamins and/or minerals that would add nutritional value to the cultured meat product.

The production of cultured meat is based upon the principle that muscle tissue can be grown from a myosatellite cell. The myosatellite cell is of non-human animal origin, preferably from non-human mammal origin, more preferably from bovine, sheep, pigs, and the like. The myosatellite cell may be obtained via a non-sacrificial and animal-friendly method, e.g. via a small biopsy. Cultured meat as referred to herein is suitable for human consumption. The modified polysaccharide hydrogel according to the present invention typically provides a suitable environment for myosatellite cell guidance, spreading, migration, proliferation and differentiation to muscle tissue. The muscle tissue may be harvested and may be sold as cultured meat.

The modified polysaccharide hydrogel according to the present invention may be produced by the provision of a modified alginate with a Mw of 10 to 50 kDa and/or a M/G ratio of 0.8 to 1.5, modified with a first specific peptide. The modification may involve a chemical coupling reaction that covalently binds the specific peptide to the alginate. For example, conventionally carbodiimide chemistry may be used for RGD-modification, herein the amine-functionality of RGD is coupled to carboxylates to form amide bonds.

Another approach to chemically couple the cell-adhesion peptides to the polysaccharide is by 1-ethyl-3-(3-dimethylaminopropyl)carbodiimide (EDC)/imidazole based coupling. Furthermore, alginates can also be oxidized to create reactive aldehyde groups, and consequently react with amine-, hydrazide, or aminooxy-terminated peptides to form imine, hydrazone or oxime bonds, respectively (Xu et al., Molecules 24 (2019) 3005).

Other approaches may include the coupling of maleimide-functionalities to carboxylates on the polysaccharide backbone. Consequently, maleimide can then readily react with thiol-groups of cysteine-comprising peptides via a Michael type addition (see e.g. Ravasco et al., Chem. Eur. J. (2019), 25, 43-59).

In a preferred embodiment, the modified polysaccharide hydrogel may be produced by the provision of a modified alginate with a Mw of 10 to 50 kDa and a M/G ratio of 0.8-1.5 modified with a first specific peptide and further crosslinked with cations. FIG. 6 shows the increased degradation and swelling of this preferred sacrificial polysaccharide hydrogel compared to alginates outside this range. Additionally, due to the faster degradation the hydrogel swelling is increased. As a result of this, satellite cells embedded inside the gel show a more spread out morphology in alginate with a Mw of 10 to 50 kDa and a M/G ratio of 0.8-1.5. The spread out morphology is accompanied by increased hydrogel compaction and myosin expression. The polysaccharide hydrogel, according to the present invention, thus typically provides a suitable environment for the regeneration process of muscle tissue.

Preferably, the modified polysaccharide hydrogel is produced by the provision of a modified alginate with a Mw of 10 to 50 kDa and a M/G ratio of 0.8 to 1.5 modified with a first specific peptide and modified with a further specific peptide. The modification may involve a chemical coupling reaction that covalently binds the specific peptide to the alginate. For example, conventional carbodiimide chemistry may be used for RGD-modification, herein the amine-functionality of RGD is coupled to carboxylates to form amide bonds.

FIG. 3 is an image of a pillar used to form a modified polysaccharide hydrogel according to the present invention. The location of the compacted hydrogel is indicated by the arrow.

FIG. 4 is a microscope image showing the alignment of myosatellite cells in a compacted hydrogel according to the present invention. The direction of the cellular alignment is indicated by the arrow.

FIG. 5 shows immunofluorescence images of the alignment of the myosatellite cells and fusion into multinucleated cells. Additionally, the expression of myosin, filamentous-actin and desmin, is included inside the RGD modified alginate hydrogels according to a preferred embodiment of the present invention. For the purpose of clarity and a concise description features are described herein as part of the same or separate embodiments, however, it will be appreciated that the scope of the invention may include embodiments having combinations of all or some of the features described.

1. A modified polysaccharide hydrogel, comprising a low molecular weight alginate having a M_{w} of 10 to 50 kDa and a M/G ratio of 0.8 to 1.5, wherein said alginate is conjugated with one or more cell-adhesion peptides, for use in cultured meat applications.

2. The modified polysaccharide hydrogel according to claim 1, said one or more cell-adhesion peptides are animal-free.

3. The modified polysaccharide hydrogel according to claim 1, wherein said cell-adhesion peptide comprises an integrin-binding ligand, which preferably comprises RGD.

4. The modified polysaccharide hydrogel according to claim 1 wherein said alginate is crosslinked by cations, preferably divalent cations, more preferably calcium (Ca²⁺) cations.

5. The modified polysaccharide hydrogel according to claim 1, wherein the concentration of the cations which are used for crosslinking is 0.05-0.5 M.

6. The modified polysaccharide hydrogel according to claim 1, wherein the concentration of the cations which are used during culture is 0-50 mM.

7. The modified polysaccharide hydrogel according to claim 1 wherein said alginate, further comprises one or more further specific peptides, different from said first specific peptide.

8. The modified polysaccharide hydrogel according to claim 7, wherein the one or more further specific peptide comprises a cell-adhesion peptide, preferably an integrin-binding ligand, which more preferably comprises RGD.

9. A method of producing a modified polysaccharide hydrogel comprising a low molecular weight alginate having a M_{w} of 10 to 50 kDa and a M/G ratio of 0.8 to 1.5, wherein said alginate is conjugated with one or more cell-adhesion peptides.

10. The method for producing a modified polysaccharide hydrogel according to claim 1 comprising the steps of:
providing a modified alginate with Mw of 10-50 kDa and M/G ratio of 0.8-1.5;
conjugate said modified alginate with said first specific peptide, using a reaction which is selected from one or more of the following: carbodiimide chemistry-based reaction; 1-ethyl-3-(3-dimethylaminopropyl)carbodiimide (EDC)/imidazole based coupling; oxidation of said alginate to create reactive aldehyde groups, and consequently react with amine-, hydrazide-, or aminooxy-terminated peptides to form imine, hydrazone or oxime bonds, respectively; coupling of maleimide-functionalities to carboxylates on the polysaccharide backbone followed by maleimide reaction with thiol-groups of cysteine-comprising peptides via a Michael type addition.

11. The method according to claim 8, further comprising a crosslinking step wherein said modified alginate is crosslinked with cations, preferably divalent cations, more preferably calcium (Ca²⁺) cations, wherein the concentration of the cations is preferably 0.05-0.5 M when used as crosslinker, and 0-50 mM during culture.

12. The method according to claim 10, further comprising a step of modification of the modified alginate with one or more further specific peptides.

13. The modified alginate obtainable by the method of claim 10.

14. The method of claim 9 wherein the alginate is a sacrificial biopolymer.
